(19) United States
(12) Patent Application Publication
(10) Pub. No.: US 2005/0287178 A1
(43) Pub. Date: Dec. 29, 2005

(54) Diagnosis and treatment of heavy gallbladder densities

(76) Inventor: Barrie L Steed, Calgary (CA)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/875,260

(22) Filed: Jun. 23, 2004

(51) Int. Cl. A61K 9/00

(52) U.S. Cl. 424/400

(57) ABSTRACT

The present invention relates to a new clinical finding termed heavy gallbladder densities. Methods to diagnose and treat heavy gallbladder densities are disclosed. Specifically, a method of rapid patient rotation and specialized ultrasound technique using conventional equipment are described. Heavy gallbladder densities may be treated or prevented using methods traditionally used to prevent and treat gallstones.

### FIELD OF THE INVENTION

The present invention relates generally to the fields of diagnostic imaging and gastroenterology. More particularly, the present invention relates to a previously unreported clinical condition of the gallbladder and biliary system, and methods to diagnose and treat the condition.

### BACKGROUND OF THE INVENTION

Right Upper Quadrant (RUQ) abdominal pain is a commonly presenting symptom of many clinical disorders including gallbladder disease, gallstones, cholecystitis, choledocholithiasis, pancreatitis, liver disease, peptic ulcer disease, biliary cysts, pulmonary disease, coronary artery disease, and irritable bowel syndrome. Many of these conditions are diagnosed using available tests, such as serology tests, routine ultrasound, cholecystography, cholescintigraphy, aspiration, biopsy, CT scan, and endoscopic retrograde cholangiopancreatography (ERCP). However, in a proportion of patients presenting with RUQ pain, the underlying cause of the RUQ pain remains unknown. In these patients, RUQ may occur daily or infrequently, may occur with eating or during the night, and may range in intensity and duration. Some patients have mild intermittent epigastric pain, while others experience sudden severe upper abdominal pain with nausea and vomiting.

It has been suggested that many patients with RUQ pain may be suffering from the effects of biliary sludge, as described by Shaffer (Current Gastroenterology Reports 2001, 3:166-173). Shaffer reports that biliary sludge is observed as a mixture of particulate matter (smaller than a typical gallstone) and bile, the particulate matter ranging in size from that of a calcium bilirubinate granule or cholesterol crystal (0.5 mm) to microlithiasis (up to 2 mm). A gallstone is defined as a solid particle larger than 2 mm in diameter.

Sludge, microlithiasis, and gallstones can all be diagnosed by routine transabdominal ultrasound examination. Biliary sludge appears on routine ultrasound as low-level echoes that layer in the dependent portion of the gallbladder without acoustic shadowing. Microlithiasis appears on routine ultrasound as a hyperechoic signal of 0.5 to 2.0 mm without a postacoustic shadow, and gallstones appear on routine ultrasound as echoes of high amplitude greater than 2 mm with postacoustic shadowing (Schaffer).

When particulate matter is present in sludge, or when microlithiasis or gallstones are present and build within the cystic duct, common bile duct or pancreatic duct, these ducts will become narrowed or blocked, leading to clinical conditions such as RUQ pain, cholecystitis, pancreatitis, and other clinical disorders. The prior art teaches treatment of gallstones by cholecystectomy or pharmacological intervention (for example with ursodeoxycholic acid). As biliary sludge and microlithiasis generally have a similar composition to gallstones (calcium bilirubinate, cholesterol) and are suggested to be pathogenically related to gallstones, the prior art also teaches similar methods (cholecystectomy, ursodeoxycholic acid) to treat patients having biliary sludge and microlithiasis. There appears to be no suggestion in the prior art to treat, based on disease processes, patients presenting with RUQ pain, or some other clinical abnormality causing concern with the gallbladder, bile duct, or pancreas sufficient to warrant diagnostic testing, but who present without biliary sludge, microlithiasis, or gallstones confirmed by routine ultrasound examination. Consequently, patients with undiagnosed relevant clinical symptoms are largely left untreated.

Moreover, many patients who have undergone cholecystectomy continue to suffer with RUQ pain, indicating that the source of the pain was not, in fact, completely attributable to gallstones, microlithiasis, or biliary sludge. These patients with undiagnosed RUQ pain are generally left untreated, as there is currently no medically explicable cause of their pain. Similarly, there are cases of acalculous cholecystitis, in which a patient undergoes cholecystectomy, but with no apparent pathology upon post-surgical examination of the gallbladder.

In addition to RUQ pain, certain patients have other clinical abnormalities which remain undiagnosed, such as elevated liver enzymes and pancreatitis.

It would, therefore, be desirable to determine the pathological cause of at least one of these persisting, unresolved conditions, and further, to determine a method to diagnose, prevent, and treat such pathology.

### SUMMARY OF THE INVENTION

It is an object of the present invention to provide an improved method to manage patients having a condition herein described and termed heavy gallbladder densities. Such patients may present with RUQ pain, or other clinical biliary, gallbladder, or pancreatic symptoms or findings that have previously been unexplainable or poorly managed.

In a first aspect, the present invention provides a method to diagnose gallbladder abnormalities in a patient comprising rapidly rotating the patient; immediately imaging the gall bladder of the patient using an imaging device; and observing any abnormalities within the gallbladder. The steps may be repeated several times to ensure any gallbladder abnormalities have been detected.

In a further embodiment, the gallbladder abnormalities are heavy gallbladder densities.

In an embodiment, the imaging device is an ultrasound apparatus. A specific ultrasound technique may be used, the technique including the steps of setting the apparatus to a light gain setting and recording a portion of the ultrasound examination using a cine loop setting.

In an embodiment, the step of rapidly rotating the patient includes: moving the patient to the steep right lateral decubitus position, followed by rapidly rotating the patient up to or beyond 180 degrees to the left lateral decubitus position; rotating the patient slowly to the left and right while in a prone position, followed by rapid rotation of the patient up to or beyond 270 degrees into the lateral decubitus position; rapid movement of the patient to a supine position from a non-supine position; rapid movement of the patient to an oblique or lateral decubitus position; rapid movement of the patient from standing or sitting to a lateral decubitus, supine, or oblique position; or rapid movement of the patient from a supine position to a right or left oblique position or through 360 degrees of rotation, returning to a supine position.

In another embodiment, the patient may be immobilized for a period of time prior to the rapid patient rotation.

In a second aspect, the invention provides a method for treating or preventing heavy gallbladder densities in a mammal comprising removal or ablation of the patient's gallbladder and/or cystic duct.

In a third aspect, the invention provides a method for treating or preventing heavy gallbladder densities in a mammal comprising administration of an agent capable of dissolving heavy gallbladder densities.

In an embodiment, the agent is an agent also capable of dissolving gallstones, and may be ursodeoxycholic acid, tauroursodeoxycholic acid, or chenodeoxycholic acid administered at a dose suitable to treat heavy gallbladder densities.

Other aspects and features of the present invention will become apparent to those ordinarily skilled in the art upon review of the following description of specific embodiments of the invention in conjunction with the accompanying figures. Where used herein, the term HGD refers to heavy gallbladder densities.

### BRIEF DESCRIPTION OF THE DRAWINGS

Embodiments of the present invention will now be described, by way of example only, with reference to the attached Figures, wherein:

FIG. 1 is a routine ultrasound image of a normal gallbladder;

FIG. 2 is a routine ultrasound image of a gallbladder containing gallstones;

FIG. 3 is a routine ultrasound image of a gallbladder containing biliary sludge;

FIG. 4 is a routine ultrasound image of a gallbladder containing microlithiasis;

FIG. 5 is a routine ultrasound image of a gallbladder containing HGD;

FIG. 6 is an ultrasound image of a gallbladder containing HGD obtained using the Rapid Patient Rotation and HGD Ultrasound Imaging techniques; and

FIG. 7 is an ultrasound image of a normal gallbladder without HGD, obtained using the Rapid Patient Rotation and HGD Ultrasound Imaging techniques.

### DETAILED DESCRIPTION

The applicant has found a previously unreported clinically relevant structure termed Heavy Gallbladder Densities (HGD), which is distinct from gallstones, microlithiasis, or biliary sludge, and is distinguishable from the aforementioned conditions using a specific ultrasound technique. Moreover, initial observations suggest that HGD are generally not coexistent with gallstones, microlithiasis, or biliary sludge. HGD are often associated with undiagnosed RUQ pain, although HGD may also exist without symptoms or apparent clinical complication. Further, administration of ursodeoxycholic acid to a patient prone to HGD has been shown to reduce the occurrence of HGD and related symptoms.

HGD are not visible during routine ultrasound examination, but can be visualized on ultrasound using specialized techniques termed “Rapid Patient Rotation” and “HGD Ultrasound Imaging”, which are described below. Following the Rapid Patient Rotation technique, and using the HGD Ultrasound Imaging technique, HGD are visible as discrete, relatively uniformly sized, non-shadowing echogenic structures of approximately 0.5 to 2 mm in diameter. HGD are heavier than bile, falling rapidly to the dependent gallbladder wall, where they are then obscured from further detection by ultrasound until the Rapid Patient Rotation technique is repeated.

Ultrasound Images

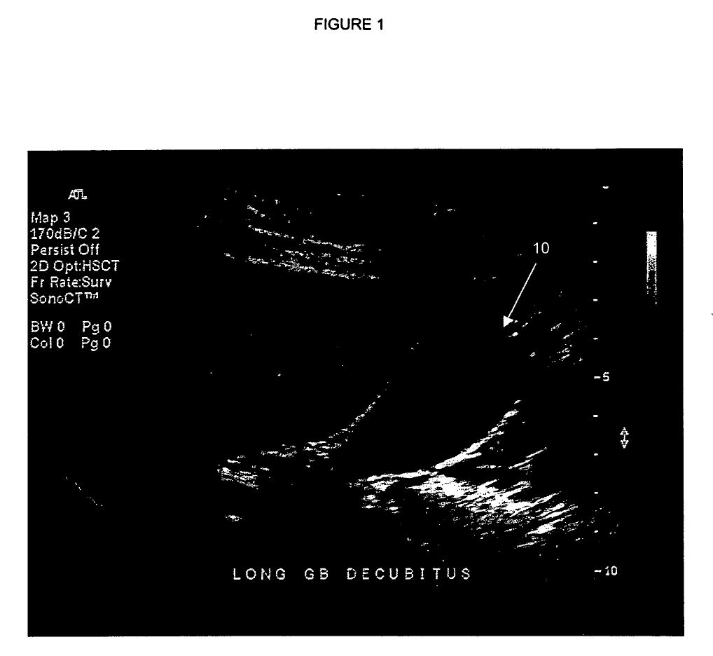

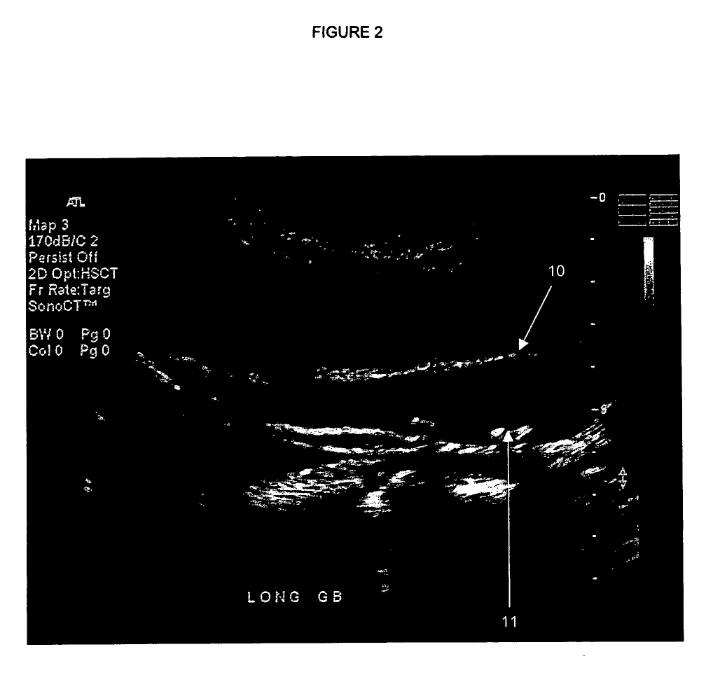

With reference to FIG. 1, in which a routine ultrasound image of a normal gallbladder 10 is shown, having no discrete structures visible within the gall bladder 10. FIG. 2 shows a routine ultrasound image of a gallbladder containing gallstones 11. A gallstone 11 appears as a bright, echogenic structure or foci on the wall of the gallbladder with acoustic shadowing beyond.

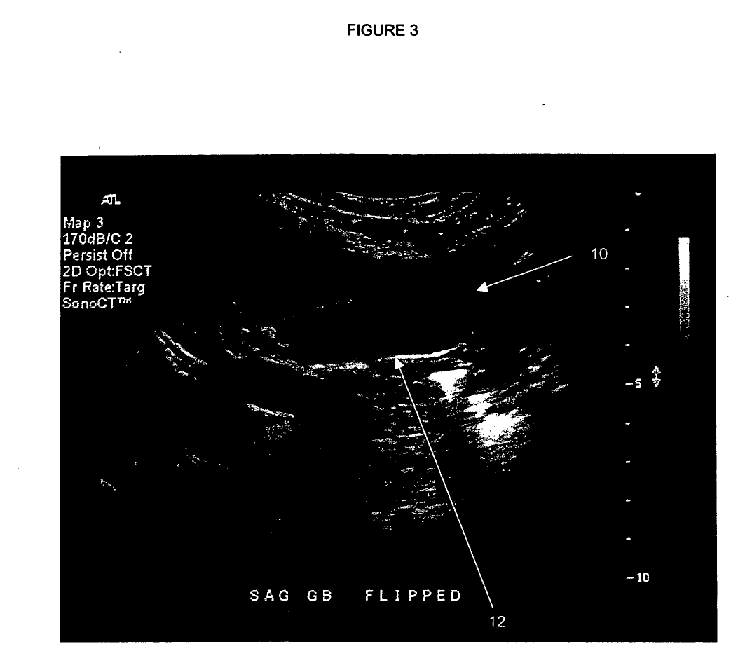

FIG. 3 is a routine ultrasound image of a gallbladder containing biliary sludge 12. The sludge is a small mass-like structure with no acoustic shadowing having low level internal echoes resting on the gallbladder wall.

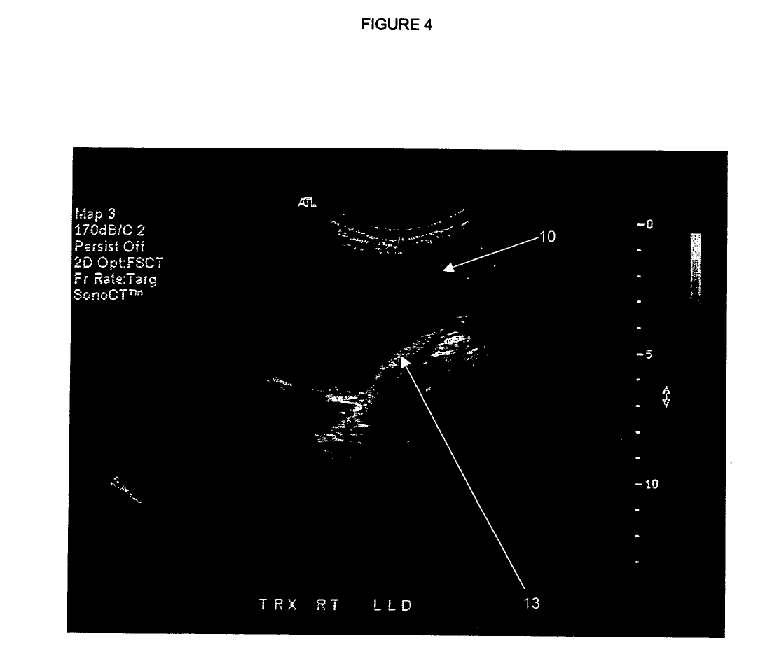

FIG. 4 is a routine ultrasound image of a gallbladder containing microlithiasis 13. Microlithiasis is visible as a bright, echogenic, non-shadowing mass-like collection on the gallbladder wall.

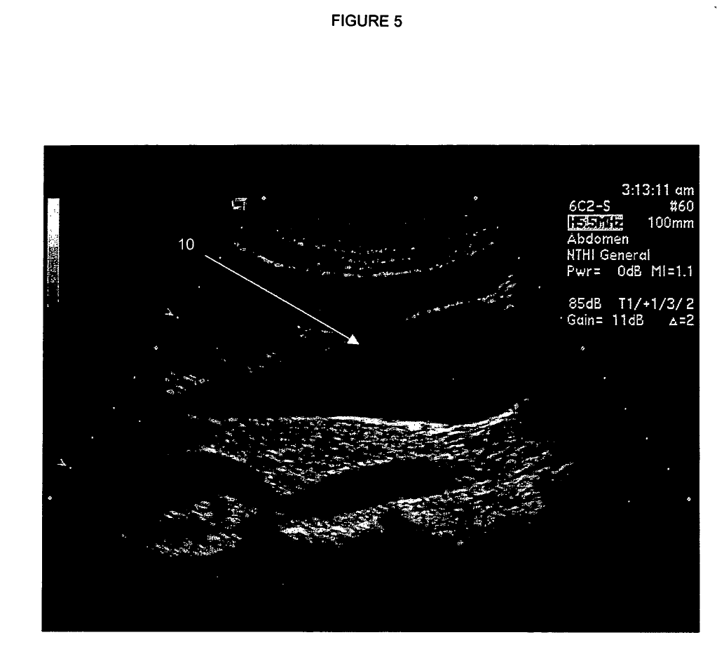

FIG. 5 is a routine ultrasound image of a patient with HGD. The image as obtained using routine ultrasound procedures appears similar to that of FIG. 1. Clearly, this image would lead a radiologist to conclude that gallstones, microlithiasis, and biliary sludge are not present within the gallbladder of this HGD patient.

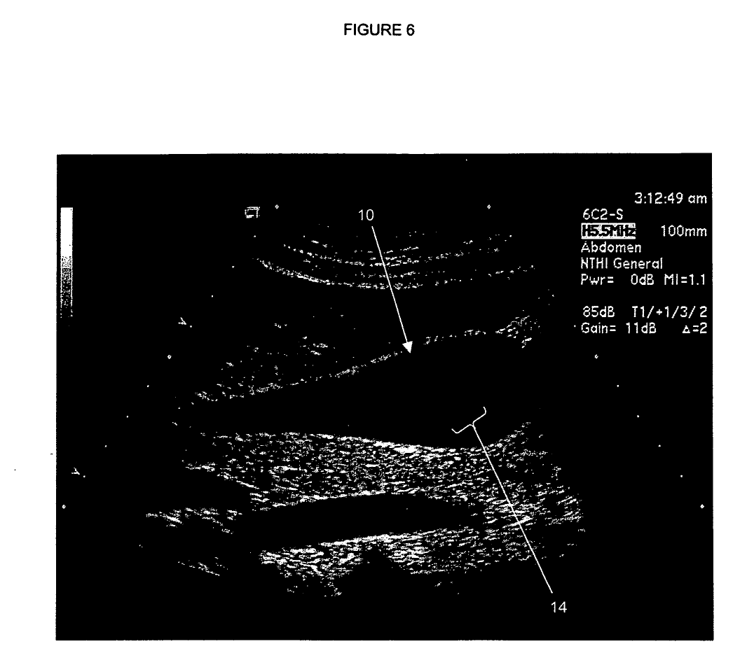

FIG. 6 is an ultrasound image of the same gallbladder shown in FIG. 5, except the image was obtained following Rapid Patient Rotation, and using the HGD Ultrasound Imaging technique. HGD 14 are temporarily visible as multiple, small bright echogenic foci within the bile of the gallbladder prior to their migration to rest against the dependent gallbladder wall, where they are not visible.

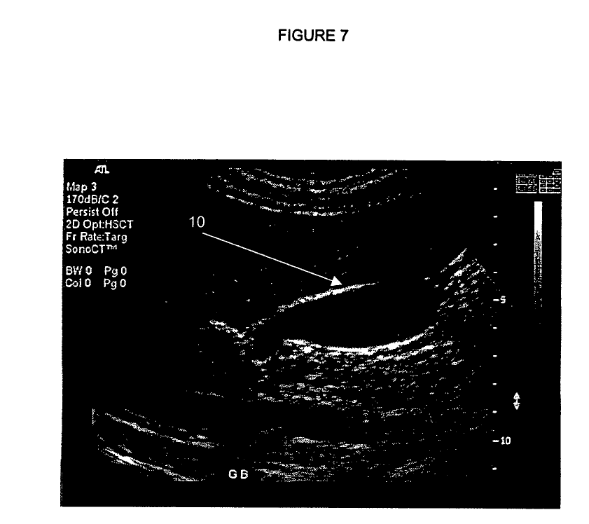

FIG. 7 is an ultrasound image of a normal gallbladder obtained following Rapid Patient Rotation, and using the HGD Ultrasound technique. HGD are not present within this image, lending support to the clinical relevance of HGD.

Rapid Patient Rotation Technique

As with a routine gallbladder ultrasound procedure, the patient should be fasting and prepped for the ultrasound procedure. As HGD are not visible when lying against the dependent gallbladder wall, the Rapid Patient Rotation technique is intended to agitate the bile to temporarily suspend the HGD within the bile, thus allowing visualization of HGD. Preferably, the patient is initially held in a stationary position or moves gently in a rocking arc-like motion to cause any HGD present to co-localize against a dependent gallbladder wall. The Rapid Patient Rotation follows, in which the patient is preferably rotated between 180 and 270 degrees in one fluid movement.

The Rapid Patient Rotation Method may take several forms, the five preferred techniques are:
1) Move the patient to the steep right lateral decubitus position, wait one minute, rapidly rotate the patient in a supine or prone direction 180 degrees to the left lateral decubitus position;
2) From the prone position, intermittently rock the patient slowly to the left and right for up to several minutes while the patient remains in a generally prone position, then rapidly rotate the patient 270 degrees into the steep lateral decubitus position, supine position, or oblique position with one side elevated from the table;
3) With the patient lying in left lateral decubitus position, rotate the patient rapidly to right lateral decubitus position, to supine position or oblique position with left or right side elevated from the table;
4) Rapid movement from a sitting or standing position to a left lateral decubitus, supine, or oblique position.

5) Rapid movement from a supine position to a right or left oblique position, or through 360 degrees, returning to a supine position.

Following Rapid Patient Rotation, the ultrasound transducer should be immediately positioned against the gallbladder, followed by imaging using the HGD Ultrasound Imaging technique described below.

HGD Ultrasound Imaging Technique

When imaging the gallbladder to detect HGD, the gallbladder should fill the image frame, with settings adjusted to a light technique for optimum brightness, as a dark technique will obscure HGD. HGD fall rapidly to the dependent gallbladder wall to become obscured from ultrasound imaging, so a cine loop may be used to record the images for later analysis.

In viewing the images, HGD are visible as small non-shadowing echogenic structures when suspended within bile. In the patients examined to date, the presence of even one HGD has been clinically significant, correlating with clinical abnormalities such as RUQ pain, pancreatitis, elevated liver enzymes, or other clinical disorders. The number of HGD present, however, does not seem to correlate with the number of abnormalities present, or the severity of any abnormality present. Therefore, detection of one or more HGD should be considered abnormal, particularly with other significant clinical findings, including those previously mentioned.

HGD are often elusive and visualization by HGD Ultrasound Imaging may require several Rapid Patient Rotation manipulations. In the appropriate clinical setting, if HGD are not visualized after several rotation attempts on the initial ultrasound, the ultrasound should be repeated, preferably up to three times at two week intervals. The majority of clinically significant HGD cases will be detectable using this protocol.

HGD Formation

Without intending to be limited to a specific mechanism, it is believed that HGD may form in the small corrugated saccule-like structures in the cystic duct, when saccules are present. A nidus likely develops in a small saccule of the cystic duct, which grows to a HGD and migrates into the gallbladder or passes into the duodenum. Although HGD could theoretically form within the gallbladder, their uniform size would favour a cystic duct saccule origin. This is supported by patient reports of continued RUQ pain following cholecystectomy, in which the gallbladder is removed, but the cystic duct remains intact. HGD may intermittently pass through the cystic duct and ampulla to restrict or obstruct bile flow, resulting in clinical manifestations. It is unlikely that HGD cause pain while in the gallbladder.

HGD are likely to be fragile and easily disintegrated, preventing their detection by ERCP or by physical manipulation and palpation of the gallbladder. It appears as though a small percentage of patients with HGD remain asymptomatic, indicating that HGD may disintegrate because of fragility, or may be small and non-obstructing. Fragility of HGD is supported by the temporary nature of the RUQ pain experienced by most patients with HGD.

Clinical relevance of HGD may result when a single or several HGD are localized within the cystic duct, ampulla, or pancreatic duct, causing obstruction. Impaction of many HGD within the ampulla or other ducts may also cause aggravated symptoms and other clinical findings.

Clinical Observations

In patients studied to date, HGD have been found in approximately 6% of all approximately 3000 gallbladders examined using Rapid Patient Rotation and HGD Ultrasound Imaging. In 200 patients with HGD, approximately 60% of the patients were female, and HGD patients were generally of average weight. Notably, patients with HGD do not display the characteristics that are generally recognized as predisposing a patient to gallstones. This supports the observation that HGD are not generally found in patients having gallstones or biliary sludge, and that HGD does not seem to be a precursor.

Most patients with HGD initially presented to their general physician with intermittent RUQ pain, and remained undiagnosed following investigations including one or more normal routine ultrasounds. Other patients had clinical findings consistent with pancreatitis or had undiagnosed elevation of liver enzymes.

Of the 200 patients diagnosed with HGD using the Rapid Patient Rotation and HGD Ultrasound Imaging techniques over a one year time interval, 144 had been referred for ultrasound due to persistent intermittent undiagnosed RUQ pain with no further clinical findings, 19 had been referred as having idiopathic pancreatitis (7 had pre-existing RUQ pain, 12 did not), 19 were referred due to undiagnosed elevation of liver enzymes (9 had pre-existing RUQ pain, 10 did not), 16 were referred for other unrelated reasons, and did not have RUQ pain, and 2 were referred as having chronic pancreatitis (both had prior RUQ pain).

Treatment of HGD-related pathology may include removal or ablation of the gallbladder and/or cystic duct, papillotomy, diet modification, ultrasound lithotripsy, or administration of gallstone dissolving agents such as ursodeoxycholic acid, chenodeoxycholic acid, tauroursodeoxycholic acid, or GS100. Moreover, certain studies have shown that gallstones may be dissolved by various combination therapies, such as ursodeoxycholic acid with lovastatin, or ursodeoxycholic acid with simvastatin. Such therapies may also be effective in treating or preventing HGD.

In an unpublished study, a 53 year old patient in which HGD were observed had an 18 year history of undiagnosed intermittent upper abdominal pain. The pain was generally exacerbated by consumption of fatty foods, with pain beginning in the epigastric area and radiating to her back and shoulders. The pain varied in length and severity, lasting from 30 minutes to 2 hours, but was not associated with any other symptoms or clinical abnormalities, although her liver enzymes were mildly elevated. Routine ultrasound appeared normal, however Rapid Patient Rotation and use of the HGD Ultrasound technique showed heavy gallbladder densities. The patient began treatment with ursodeoxycholic acid (250 mg QAM, 500 mg QPM with food) and became asymptomatic within three weeks. A repeat ultrasound (using Rapid Patient Rotation and HGD Ultrasound technique) three months later was unable to detect HGD.

After several months of treatment, the patient stopped taking ursodeoxycholic acid, at which time she experienced recurrence of the sporadic upper abdominal pain. Subsequent ursodeoxycholic acid treatment again resulted in complete resolution of the pain.

In a second unpublished case study, a 52 year old female had experienced right upper quadrant and epigastric pain for approximately 10 years. She had been treated for gastric reflux, which alleviated her symptoms to some extent, however the RUQ pain continued sporadically with variable duration (up to 12 hours) and intensity. Her liver enzymes were mildly elevated, however no other clinical abnormalities were apparent.

Rapid Patient Rotation and HGD Ultrasound technique indicated the presence of HGD, following which she was treated with ursodeoxycholic acid (250 mg QAM, 500 mg QPM with food), becoming nearly asymptomatic within three weeks. Failure to take ursodeoxycholic acid resulted in recurrence of the RUQ pain.

The above-described embodiments of the present invention are intended to be examples only. Alterations, modifications and variations may be effected to the particular embodiments by those of skill in the art without departing from the scope of the invention, which is defined solely by the claims appended hereto.

1. A method to diagnose gallbladder abnormalities in a patient comprising the steps:
a. rapidly rotating the patient;
b. immediately imaging the gall bladder of the patient using an imaging device; and
c. observing any abnormalities within the gallbladder.

2. The method of claim 1 wherein the gallbladder abnormalities are heavy gallbladder densities.

3. The method of claim 1 further comprising a step (d) of repeating steps (a) through (c) until a gallbladder abnormality is observed.

4. The method of claim 1 wherein the imaging device is an ultrasound apparatus.

5. The method of claim 4 wherein a specific ultrasound technique is used, the technique including the steps of setting the apparatus to a light gain setting and recording a portion of the ultrasound examination using a cine loop setting.

6. The method of claim 1 wherein step (a) includes moving the patient to the steep right lateral decubitus position, followed by rapidly rotating the patient 180 degrees to the left lateral decubitus position.

7. The method of claim 1 wherein step (a) includes intermittently rotating the patient to the left and right while in a prone position, followed by rapid rotation of the patient 270 degrees into the lateral decubitus position.

8. The method of claim 1 wherein step (a) includes rapid movement of the patient to a supine position from a non-supine position.

9. The method of claim 1 wherein step (a) includes rapid movement of the patient to an oblique or lateral decubitus position.

10. The method of claim 1 wherein step (a) includes rapid movement of the patient from a standing or sitting position to a supine, oblique, or lateral decubitus position.

11. The method of claim 1 wherein step (a) includes rapid movement of the patient from a supine position to a left or right oblique position, or through 360 degrees of rotation, returning to a supine position.

12. The method of claim 1 further comprising the step of immobilizing the patient for a period of time prior to step (a).

13. A method for treating or preventing heavy gallbladder densities in a mammal comprising removal or ablation of the patient's gallbladder.

14. A method for treating or preventing heavy gallbladder densities in a mammal comprising removal or ablation of the patient's cystic duct.

15. A method for treating or preventing heavy gallbladder densities in a mammal comprising administration of an agent capable of dissolving heavy gallbladder densities.

16. The method of claim 15 wherein the agent is an agent capable of dissolving gallstones.

17. The method of claim 16 wherein the agent comprises a member selected from the group consisting of ursodeoxycholic acid, chenodeoxycholic acid, and tauroursodeoxycholic acid.
